(19) United States
(12) Patent Application Publication
(10) Pub. No.: US 2004/0039237 A1
(43) Pub. Date: Feb. 26, 2004

(54) Stabilized trichloroethane

(76) Inventors: Earl M. Gorton, Sulphur, LA (US); Ronald D. Olinger, Lake Charles, LA (US)

Correspondence Address: PPG INDUSTRIES, INC., Intellectual Property, One PPG Place, Pittsburgh, PA, 15272, US

(21) Appl. No.: 10/648,972

(22) Filed: Aug. 27, 2003

Related U.S. Application Data
(63) Continuation-in-part of application No. 10/436,664, filed on May 13, 2003.
(60) Provisional application No. 60/396,460, filed on Jul. 16, 2002.

(51) Int. Cl. C07C 17/42

(52) U.S. Cl. 570/104

(57) ABSTRACT

Trichloroethane, e.g., 1,1,1-trichloroethane, is stabilized with a stabilizing amount of a stable free radical stabilizer, e.g., a material having a 2,2,6,6-tetra (lower alkyl)-1-piperidinyloxy-yl free radical group such as 2,2,6,6-tetramethyl-4-hydroxy-1-piperidinyloxy.

### CROSS REFERENCE TO RELATED APPLICATIONS

This application is a continuation-in-part application of Ser. No. 10/436,664 filed May 13, 2003, which application claims priority to U.S. provisional patent application Serial No. 60/396,460 filed Jul. 16, 2002.

### DESCRIPTION OF THE INVENTION

The present invention relates to the stabilization of trichloroethane. In particular, this invention relates to the stabilization of trichloroethane, e.g., 1,1,1-trichloroethane and its isomer 1,1,2-trichloroethane, during storage and shipment. More particularly, this invention relates to trichloroethane containing a stabilizing amount of stable free radical stabilizer, e.g., a material having a 2,2,6,6-tetra (lower alkyl)-1-piperidinyloxy-yl free radical group.

Other than in any operating examples, or where otherwise indicated all numbers expressing quantities that are used in this specification and the accompanying claims are to be understood as modified in all instances by the term “about”.

### DETAILED DESCRIPTION OF THE INVENTION

1,1,1-Trichloroethane (viz., methyl chloroform) is commonly produced commercially by reacting 1,1-dichloroethane and molecular chlorine in the liquid phase and in the presence of free radical initiator. Similarly, the addition of molecular chlorine to chloroethene (viz., vinyl chloride) in the liquid phase to produce 1,1,2-trichloroethane is known. This latter reaction may proceed by an ionic path when a metal catalyst such as FeCl₃ is used, or by a radical path. See, for example, Kirk-Othmer Encyclopedia of Chemical Technology, third edition, volume 23, John Wiley & Sons, New York (1983), page 868, and Chemical Abstracts, volume 47, American Chemical Society, Columbus Ohio (1953), column 11218f abstracting JP 26[1951]-6873. U.S. Pat. No. 6,150,573 discloses a method for the concurrent production of 1,1,1-trichloroethane and 1,1,2-trichloroethane by feeding molecular chlorine, chloroethene and 1,1-dichloroethane to a reaction vessel.

Trichloroethane, e.g., 1,1,1-trichloroethane (viz. methyl chloroform), is a normally liquid chlorinated hydrocarbon, which has utility as an industrial solvent and as a feed stock useful in the preparation of other halogenated hydrocarbons, e.g., fluorinated hydrocarbons and chloro-fluoro hydrocarbons. Trichloroethane suffers from the disadvantage of being unstable, particularly in the presence of air, water, metals, such as aluminum, magnesium, zinc, iron, copper and their alloys, and various metal halides, particularly the Friedel-Crafts metal halides, e.g. aluminum chloride, which catalyze the decomposition reaction. When undergoing decomposition, trichloroethane will split off hydrogen chloride gas and then blacken due to the formation of other decomposition products. The decomposition reaction can progress to the complete resinification of the material.

It has, therefore, been customary to add small amounts of one or more stabilizer materials in order to reduce the tendency of trichloroethane, particularly methyl chloroform, to decompose. Some of such stabilizer materials are intended to stabilize vaporous trichloroethane, while some are intended to stabilize liquid trichloroethane. In some cases, the stabilizer material is used to stabilize both vaporous and liquid trichloroethane, depending on the use for which the trichloroethane is intended. Some stabilizer materials reduce the susceptibility of trichloroethane to metal induced decomposition, while others serve as an acceptor for the acid, e.g., hydrochloric acid, which is produced during decomposition.

Examples of stabilizer compounds that have been described in the patent literature for use as materials to stabilize methyl chloroform include aliphatic alcohols, nitroalkanes, dialkoxyalkanes, epoxides, nitrites, ketones, dioxolane, ketone alcohols, dialkyl sulfoxides, acetaldehyde and dimethyl hydrazone. See, for example, U.S. Pat. No. 4,394,284, which describes in column 1 the wide variety of stabilizers used in combination with nitroalkanes to stabilize methyl chloroform. See also, U.S. Pat. Nos. 3,251,891, 3,265,741, 3,281,480, 3,499,047, 3,532,761, 3,535,392, 4,018,837, 4.069.265. 4,309,301, 4,324,928, 4,351,973 and 4,992,604, which illustrate various combinations of the aforedescribed materials as stabilizers for methyl chloroform. The aforedescribed stabilizers and combination of stabilizers have been proposed to stabilize methyl chloroform for its proposed use as an industrial solvent, e.g., as a liquid and Vapor phase degreasing solvent for metals, such as aluminum. However, more recently methyl chloroform has been used as a feed stock for the preparation of chloro-fluoro hydrocarbons materials. In this more recent application, it is important to minimize the number and amount of stabilizer materials used to stabilize methyl chloroform so as not to interfere with the chemical process, e.g., deactivate the catalyst, used to prepare such chloro-fluoro hydrocarbon materials.

In our copending application Ser. No. 10/436,664 filed May 13, 2003, stable free radical stabilizer is described for stabilizing trichloroethane during processing at temperatures at which trichloroethane is susceptible to thermal decomposition, e.g., during distillation. However, because of the difference in boiling points of the free radical stabilizer, e.g., 4-hydroxy-TEMPO (2,2,6,6-tetrametlhyl-4-hydroxy-1-piperidinyloxy), and trichloroethane, the higher boiling free radical stabilizer is withdrawn with the bottoms stream removed from the distillation column and is not removed with product trichloroethane removed as distillate from the distillation column.

It has now been surprisingly discovered that trichloroethane; namely, 1,1,1-trichloroethane and/or 1,1,2-trichloroethane, can be stabilized against air oxidation during storage and transportation by the presence of a stabilizing amount of a stable free radical material. An example of a type of stable free radical found to be particularly effective is characterized as having at least one 2,2,6,6-tetra(lower alkyl)-1-piperidinyloxy-yl free radical group. Moreover, it is contemplated that such free radical stabilizer will not adversely affect, e.g., deactivate, the catalyst used by manufacturers that use methyl chloroform as a feed stock for the production of fluorinated hydrocarbons.

As is known in the art, 1,1,1-trichloroethane is typically produced by introducing 1,1-dichloroethane, molecular chlorine and free radical initiator into a suitable reactor that contains a liquid reaction mixture. The reactants may be introduced as separate streams or two or more of the reactants may be combined prior to introduction. In many cases only a portion of the molecular chlorine introduced is available for the desired chlorination. This may be due to a variety of causes, such as undesired side reactions and loss through the various outlets. It may be seen that the availability of chlorine atoms for the desired chlorination is a factor to be considered in choosing the relative proportions of molecular chlorine and organic feedstock to be used in conducting the reaction. In general, sufficient molecular chlorine is introduced into the reactor to accomplish the desired degree of chlorination of the feedstock. Usually, but not necessarily, the mole ratio of molecular chlorine to 1,1-dichloroethane charged to the reactor is in the range of from 0.3:1 to 2.5:1. Often the ratio is in the range of from 0.4:1 to 2:1, e.g., from 0.5:1 to 2:1.

Free radical initiators that can be used in the production of 1,1,1-trichloroethane by the aforedescribed process are numerous and widely varied. See, for example, Kirk-Othmer Encyclopedia of Chemical Technology, third edition, volume 17, pages 1-90 (1982). In most cases, organic free radical initiators are used. One class of suitable organic free radical initiators comprises organic peroxygen-containing free radical initiators. This class of initiators can be divided into a large number of subclasses, some of which include aliphatic peroxides, e.g., di-tert-butyl peroxide [CAS 110-05-4]; hydroperoxides, e.g., tert-butyl hydroperoxide [CAS 75-91-2]; ketone peroxides, e.g., methyl ethyl ketone peroxide; aldehyde peroxides, e.g., bis (1-hydroxyheptyl) peroxide; diperoxyketals, e.g., 2,2-bis (tert-butylperoxy) butane [CAS 2167-23-9]; diperoxyketals, e.g., 2,2-bis (tert-butylperoxy) butane [CAS 2167-23-9]; diacyl peroxides, e.g., diacetyl peroxide [CAS 110-22-5], dibenzoyl peroxide [CAS 94-36-0], and diisobutyryl peroxide [CAS 3437-84-1]; peroxycarboxylic acids, e.g., peroxyacetic acid; peroxyesters, e.g., tert-butyl peroxyacetate [CAS 107-71-1], peroxycarbonates, e.g., tert-butylperoxy isopropyl carbonate; and peroxydicarbonates, e.g., diisopropyl peroxydicarbonate [CAS 105-64-6], di-sec-butyl peroxydicarbonate, di-n-propyl peroxydicarbonate [CAS 16066-38-9], di(2-ethylhexyl) peroxydicarbonate, dicyclohexyl peroxydicarbonate [CAS 1561-49-5], and dicetyl peroxydicarbonate [CAS 26322-14-5]; and organic azonitrile initiators, e.g., 2,2′-azobis(2-methylpropanenitrile) [CAS 78-67-1]. Additional examples of free radical initiators can be found in our copending application Ser. No. 10/436,664 filed May 13, 2003, which examples are incorporated herein by reference.

The amount of free radical initiator present in the liquid reaction mixture during the reaction can vary widely. The amount introduced depends upon many factors including, but not limited to: the identity and activity of the initiator; the composition of the organic feedstock; and the presence, identity, and concentrations, if any, of free radical poisons or inhibitors. In general, the free radical initiator is present in the liquid reaction mixture in at least an initiating amount. The minimum and maximum amounts are not limited by any theory, but by practical convenience. In most instances, the ratio of the weight of free radical initiator introduced to the reactor to the sum of the weight of the hydrocarbon reactant, e.g., 1,1-dichloroethane, or 1,1-dichloroethane and chloroethene, introduced into the reactor is in the range of from 50 to 5000 parts per million parts (ppm). Often the ratio is in the range of from 75 to 3000 ppm, e.g. from 100 to 1000 ppm.

The temperature at which the liquid phase chlorination is conducted can vary considerably. Usually, but not necessarily, the temperature is in the range of from 60° C. to 140° C., e.g., a temperature in the range of from 90° C. to 120° C. The pressure at which the liquid phase chlorination is conducted can also vary widely. It may be subatmospheric, ambient atmospheric or superatmospheric. In most cases., it is at about ambient atmospheric pressure or somewhat higher. In many instances the pressure is in the range of from 0 to 1400 kilopascals, gauge. Often the pressure is in the range of from 100 to 1000 kilopascals, gauge, e.g., in the range of from 340 to 850 kilopascals, gauge.

Hydrogen chloride is removed from the reactor, usually as a gas, while the organic reaction product(s) can be removed from the reactor as a liquid or as a gas. In most instances the organic reaction product is removed as a liquid, but it may be vaporized and removed as a gas.

The organic reaction product removed from the reactor can be further processed as desired. In most cases, it is forwarded to a purification zone where the desired components are recovered as purified product compounds. The purification zone usually comprises a train of distillation columns. In a conventional purification zone, the organic reaction product removed from the reactor is forwarded to a first distillation column. An overhead stream comprising chiefly unreacted 1,1-dichloroethane is removed from or near the top of the first distillation column and is recycled to the reactor. A bottoms stream from the first distillation column comprising chiefly 1,1,1-trichloroethane, 1,1,2-trichloroethane, 1,2-dichloroethane and heavies is removed from or near the bottom of the first distillation column and is forwarded to a second distillation column. An overhead product stream comprising predominately 1,1,1-trichloroethane and some 1,2-dichloroethane is removed from or near the top of the second column. A bottoms stream comprising chiefly 1,1,2-trichloroethane and heavies is removed from or near the bottom of the second distillation column and is forwarded for further purification.

An example of stable free radical stabilizer material that can be used to stabilize trichloroethane is a material that has at least one 2,2,6,6-tetra (lower alkyl)-1-piperidinyloxy-yl free radical group. The lower alkyl groups can be the same or they may be different, but usually they will be the same, and will comprise from 1 to 5, e.g., 1 to 4, carbon atoms. The lower alkyl group usually employed is methyl or ethyl, although lower alkyl groups having more than two carbon atoms, e.g., three or four carbon atoms, are contemplated. Typically, the lower alkyl group is methyl.

The 2,2,6,6-tetra(lower alkyl)-1-piperidinyloxy-yl free radical group is usually the 2,2,6,6-tetra(lower alkyl)-1-piperidinyloxy-4-yl free radical group, but the 2,2,6,6-tetra(lower alkyl)-1-piperidinyloxy-3-yl free radical group may be used, if desired, The 2,2,6,6-tetra(lower alkyl)-1-piperidinyloxy-yl free radical group can be attached to hydrogen, hydroxyl, oxo, or to a parent compound as a substituent. In those embodiments in which the stable free radical is substituted onto a parent compound, the typical parent compound is a monocarboxylic acid or a dicarboxylic acid, in which case the stable free radical stabilizer material is an ester. The monocarboxylic acids can be aliphatic or aromatic. In one contemplated embodiment, the aliphatic monocarboxylic acid is saturated and contains from 1 to 18 carbon atoms. In other contemplated embodiments, the aliphatic monocarboxylic acid contains from 2 to 12 carbon atoms, e.g., from 3 to 8 carbon atoms. Of the aromatic monocarboxylic acids, benzoic acid is a particular embodiment. When dicarboxylic acids are used as the parent compound, the dicarboxylic acids can be saturated and contain from 2 to 13 carbon atoms. In one contemplated embodiment, the saturated dicarboxylic acid contains from 4 to 12 carbon atoms, e.g., from 8 to 12 carbon atoms. A particular contemplated embodiment of a saturated dicarboxylic acid is sebacic acid, which contains 10 carbon atoms. It should be understood that the stable free radical material of the present invention need not be associated with a parent compound, and in embodiments of the present invention, the stable free radical material itself is used.

Stable free radicals described herein and methods for their preparation are known to those skilled in the art. Non-limiting examples of suitable free radical materials that can be used in the present invention include:

2,2,6,6-tetramethyl-1-piperidinyloxy [CAS 2564-83-2];

2,2,6,6-tetramethyl-4-hydroxy-1-piperidinyloxy [CAS 2226-96-2] having the structure:

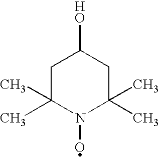

which material is also known as 4-hydroxy-TEMPO, and which is commercially available as a 5% active ingredient in an inert solvent mix from GE Betz as PETROFLO 20Y104, and which is also available in solid form from Ciba Specialty Chemicals as PROSTAB 5198;

2,2,6,6-tetramethyl-4-oxo-1-piperidinyloxy [CAS 2896-70-0];

2,2,6,6-tetramethyl-4-amino-piperidinyloxy;

2,2,6,6-tetramethyl-4-dimethylamino piperidinyloxy;

2,2,6,6-tetramethyl-4-ethanoyloxy piperidinyloxy;

2,2,6,6-tetramethyl-4-((methylsulfonyl)oxy)-1-piperidinyloxy [CAS 35203-66-8];

2,2,6,6-tetramethyl-1-piperidinyloxy-4-yl benzoate [CAS 3225-26-1]; and

bis(2,2,6,6-tetramethyl-1-piperidinyloxy-4-yl) sebacate [CAS 2516-92-9], which is available commercially in solid form and as a 4 to 10% solution in an organic solvent from Ciba Specialty Chemicals as PROSTAB 5415.

Other examples of stabilizer materials that are contemplated for use as a stabilizer for trichloroethane include materials having a 2,2,5,5-tetra(lower alkyl) pyrrolidinyloxy group. As in the case of the 2,2,6,6-tetra(lower alkyl)-1-piperidinyloxy-yl group described heretofore, the lower alkyl groups can be the same or different, but usually will be the same, and will comprise from 1 to 5, e.g., 1 to 4, carbon atoms. The lower alkyl group usually employed is methyl or ethyl. Typically, the lower alkyl group is methyl, i.e., 2,2,5,5-tetramethyl pyrrolidinyloxy. Non-limiting examples of such stabilizer materials include:

2,2,5,5-tetramethyl-pyrrolidinyloxy;

3-amino-2,2,5,5-tetramethyl-pyrrolidinyloxy;

2,2,5,5-tetramethyl-1-oxa-3-azacyclopentyl-3-oxy; and

2,2,5,5-tetramethyl-3-pyrrolinyl-1-oxy-3-carboxylic acid.

The amount of stable free radical stabilizer additive that is used to stabilize trichloroethane, as described herein, can vary. In general, the amount of stable free radical stabilizer additive used can be characterized as a stabilizing amount. It is contemplated that at least one stable free radical stabilizer material, such as the free radical stabilizer materials described above, derivatives thereof, or equivalents of such stabilizer materials or derivatives thereof can be used. The amount of stable free radical additive added to the process stream will also depend upon the degree of stability desired and the effectiveness of the particular stable free radical employed. Minimal amounts may yield de minimis improvements. On the other hand diminishing returns are generally encountered when using amounts significantly larger than the amounts that provide an economically effective deterrent to air induced decomposition, i.e., the formation of undesired levels of contaminating decomposition products. The upper and lower limits of practical effectiveness can be readily determined by one skilled in the art by measuring the amounts of decomposition products in the trichloroethane as the amount of stable free radical additive added to the trichloroethane is varied. Another useful method for measuring the effectiveness of the free radical stabilizer and the amounts thereof used is to measure over time the pH of trichloroethane containing various amounts of stable free radical stabilizer.

It is contemplated that stable free radical stabilizer will be present in amounts of at least 5 parts per million parts (ppm) of trichloroethane, e.g. methyl chloroform, e.g., from 5 to 20 ppm. In a particular contemplated embodiment, it is contemplated that from 5 ppm to 15 ppm, of stable free radical stabilizer is used. T he amount of stable free radical stabilizer used can vary in amounts ranging between any of the aforedescribed upper and lower values, inclusive of the recited values.

As noted earlier, stabilization of trichloroethane containing free radical stabilizer material(s) can be measured by measuring the pH (acidity) over time of the trichloroethane with and without use of one or more stable free radical stabilizers. The following methods for determining the pH of trichloroethane can be used, which methods were the methods followed in the Example. The described methods are similar except for amounts of reagent water and sample that are used.

The following method was used to obtain the initial and final pH measurements of the test samples:

Add 75 milliliters (mL) of reagent water containing sodium chloride to a 250 mL glass beaker, and adjust the pH of the water to 7.0 with either 0.01 N sodium hydroxide or 0.01 N hydrochloric acid, as required. The reagent water is prepared by adding 0.2 to 0.4 grams of sodium chloride salt to approximately 900 mL of distilled water. This salt solution is boiled vigorously to remove carbon dioxide. After boiling, the salt solution is stoppered and allowed to cool.

With stirring, add 25±0.1 mL of the trichloroethane sample into the 250 mL beaker. Read the pH of the water-solvent emulsion immediately after the last of the trichloroethane sample is added to the beaker with a pH mete, e.g., a Thermo-Orion pH meter or equivalent.

The following method was used to obtain the pH measurements of other than the initial and final measurements of the samples: Add 4 ml of reagent water having a pH of 7.0 to a test tube. Add 1 mL of trichloroethane solvent into the test tube and shake the test tube for approximately 1 minute. Allow the organic and aqueous phases to separate in the test tube, and measure the pH of the aqueous (upper) phase with a pH meter, such as a Thermo-Orion pH meter or equivalent.

It has also been discovered that stable free radical stabilizer added to trichloroethane to stabilize it against air oxidation during storage and shipment can be removed from the trichloroethane by contacting the stabilized trichloroethane with silica, e.g., precipitated silica, silica gel and fumed silica. For economic reasons, it is contemplated that precipitated silica will typically be used. Removal of the stable free radical stabilizer from trichloroethane, e.g., methyl chloroform, may be desirable when the trichloroethane is used as a feed stock for the production of other halogenated compounds, e.g., fluorinated hydrocarbons or fluoro-chloro hydrocarbons, and a very pure trichloroethane feed stock is required.

The amount of silica required will, of course, vary and will depend on the amount of free radical stabilizer used with the trichloroethane and the adsorptive capacity of the particular silica used. Typically, an amount of silica that is sufficient to adsorb substantially all of the free radical stabilizer material in the trichloroethane composition is used. Generally, the trichloroethane will contain minor amounts of free radical stabilizer, e.g., less than 50 ppm, more usually not more than 20 ppm. In one contemplated embodiment, at least 0.05 weight percent, e.g., at least 0.1 weight percent of silica, such as precipitated silica, (based on the amount of trichloroethane treated) is sufficient to remove (adsorb) substantially all of the stable free radical stabilizer material from the trichloroethane composition, e.g., a liquid trichloroethane composition, comprising trichloroethane and 15 ppm of 4-hydroxy TEMPO free radical stabilizer. thereby to provide a liquid trichloroethane product that is substantially free of the stable free radical stabilizer.

Treatment of the trichloroethane with the silica can be performed by any convenient method and in any conventional equipment that is used to bring a liquid and solid into intimate contact. It is contemplated that the treatment will be performed under ambient conditions, and under conditions that prevents the trichloroethane from coming into significant contact with air or other destabilizing conditions, e.g., contact with water, metals, metal halides etc.

The present invention is further described in the following example, which is to be considered as illustrative, rather than limiting, of the invention, and wherein all parts are parts by weight and all percentages are percentages by weight unless specified otherwise.

### EXAMPLE

A gallon of unstabilized 1,1,1-trichloroethane containing no stabilizers was obtained from the 1,1,1-trichloroethane-production unit of PPG Industries Inc. plant at Lake Charles, La., and divided into four-quart glass amber containers. The first quart sample contained approximately 1115 grams of 1,1,1-trichloroethane, and was identified as Sample A (control—no stabilizer material added). The second quart sample contained 1096 grams of 1,1,1-trichloroethane and 32.0 grams of 1,3-dioxolane, and was identified as Sample B. The third quart sample contained 1131.2 grams of 1,1,1-trichloroethane and 0.06 grams of PROSTAB® 5415 available from Ciba Specialty Chemicals, and was identified as Sample C. The fourth quart sampled contained 1119.0 grams of 1,1,1-trichloroethane and 0.56 grams of butylene oxide, and was identified as Sample D. All four-quart samples were capped and placed in a convection oven that was maintained at 120° F.(48.8° C.) No precautions were made to exclude air from the oven. From time to time over a period of one year, the pH of the samples was measured using the methods described above. The pH measurements obtained are tabulated in Table I.

TABLE I

| Date | Sample A | Sample B | Sample C | Sample D |
|---|---|---|---|---|
| 10/21/99 | Start | Start | Start | Start |
| 10/25/99 | 7.1 | 4.5 | 7.2 | 7.2 |
| 11/10/99 | 7.15 | | 7.2 | 7.2 |
| 11/16/99 | 7.15 | | 7.1 | 7.15 |
| 11/23/99 | 7.1 | | 7.15 | 7.0 |
| 11/29/99 | 7.0 | | 6.95 | 7.0 |
| 12/07/99 | 6.8 | | 7.2 | 7.2 |
| 12/15/99 | 4.0 | | 6.9 | 6.9 |
| 12/20/99 | 3.6 | | 6.8 | 6.8 |
| 01/03/00 | | | 7.1 | 6.9 |
| 01/11/00 | | | 7.0 | 7.05 |
| 01/17/00 | | | 7.2 | 7.1 |
| 02/07/00 | | | 7.05 | 7.0 |
| 02/29/00 | | | 6.9 | 6.95 |
| 04/07/00 | | | 7.1 | 7.2 |
| 05/05/00 | | | 7.1 | 7.1 |
| 05/26/00 | | | 6.95 | 7.05 |
| 06/15/00 | | | 7.1 | 7.2 |
| 07/07/00 | | | 6.95 | 7.0 |
| 08/25/00 | | | 7.0 | 6.95 |
| 10/06/00 | | | 6.95 | 6.9 |

The data of Table I show that 1,1,1-trichloroethane stabilized with 1,3-dioxolane (Sample B) decomposed after 4 days, and the control sample (Sample A) decomposed after about 2 months, as shown by the acid pH of the samples. The samples stabilized with PROSTAB 5415 (Sample C) and butylene oxide (Sample D) were still stable after approximately one year.

Although the present invention has been described with reference to specific details of certain embodiments thereof, it is not intended that such details should be regarded as limitations upon the scope of the invention except insofar as they are included in the accompanying claims.

What is claimed is:

1. A composition comprising trichloroethane and a stabilizing amount of stable free radical stabilizer.

2. The composition of claim 1 wherein the trichloroethane is selected from 1,1,1-trichloroethane, 1,1,2-trichloroethane and mixtures of 1,1,1-trichloroethane and 1,1,2-trichloroethane.

3. The composition of claim 2 wherein the free radical stabilizer is present in amounts of at least 5 parts per million parts of trichloroethane.

4. The composition of claim 3 wherein the free radical stabilizer is a material having the 2,2,6,6-tetra(lower alkyl)-1-piperidinyloxy-yl free radical group.

5. The composition of claim 4 wherein the 2,2,6,6-tetra(lower alkyl)-1-piperidinyloxy-yl free radical group is a 2,2,6,6-tetramethyl-1-piperidinyloxy-yl free radical group.

6. The composition of claim 3 wherein the stable free radical stabilizer is a material having the 2,2,6,6-tetra(lower alkyl)-1-piperidinyloxy-4-yl free radical group.

7. The composition of claim 6 wherein the stable free radical stabilizer is a material having the group 2,2,6,6-tetramethyl-4-hydroxy-1-piperidinyloxy, 2,2,6,6-tetramethyl-4-amino-piperidinyloxy, 2,2,6,6-tetramethyl-4-dimethylamino-piperidinyloxy, 2,2,6,6-tetramethyl-4-ethanoyloxy-piperidinyloxy, 2,2,6,6-tetramethyl-4-oxo-1-piperidinyloxy, 2,2,6,6-tetramethyl-4-((methylsulfonyl)oxy)-1-piperidinyloxy, or 2,2,6,6-tetramethyl-1-piperidinyloxy-4-yl benzoate.

8. The composition of claim 6 wherein the stable free radical stabilizer is bis(2,2,6,6-tetramethyl-1-piperidinyloxy-4-yl) ester of saturated dicarboxylic acid.

9. The composition of claim 8 wherein the saturated dicarboxylic acid contains from 2 to 13 carbon atoms.

10. The composition of claim 8 wherein the stable free radical stabilizer is bis(2,2,6,6-tetramethyl-1-piperidinyloxy-4-yl) sebacate.

11. The composition of claim 3 wherein the free radical stabilizer is a material having the 2,2,6,6-tetramethyl-4-hydroxy-1-piperidinyloxy group, which material is present in amounts of from 5 to 20 parts per million parts of trichloroethane.

12. The composition of claim 2 wherein the free radical stabilizer is present in amounts of from 5 to 20 parts per million parts of trichloroethane.

13. The composition of claim 3 wherein the free radical stabilizer is a material having a 2,2,5,5-tetra(lower alkyl) pyrrolidinyloxy group.

14. The composition of claim 13 wherein the 2,2,5,5-tetra(lower alkyl) pyrrolidinyloxy group is 2,2,5,5-tetramethyl pyrrolidinyloxy.

15. The composition of claim 14 wherein the free radical stabilizer is a material having the group 2,2,5,5-tetramethyl-3-amino-pyrrolidinyloxy, 2,2,5,5-tetramethyl-1-oxa-3-azacyclopentyl-3-oxy, or 2,2,5,5-tetramethyl-3-pyrrolinyl-1-oxy-3-carboxylic acid.

16. The composition of claim 15 wherein the free radical stabilizer is present in amounts of from 5 to 20 parts per million parts of trichloroethane.

17. A method for removing stable free radical stabilizer from a liquid composition comprising trichloroethane and minor amounts of stable free radical stabilizer, comprising contacting said liquid composition with an amount of silica sufficient to adsorb stable free radical stabilizer and provide a trichloroethane composition substantially free of stable free radical stabilizer.

18. The method of claim 17 wherein the silica is precipitated silica, silica gel or fumed silica.

19. The method of claim 17 wherein the silica is precipitated silica, which is used in amounts of at least 0.05 weight percent, based on the amount of trichloroethane in the liquid composition.
